(19) United States
(12) Patent Application Publication
(10) Pub. No.: US 2003/0236517 A1
(43) Pub. Date: Dec. 25, 2003

(54) Endovascular treatment device with a protective sleeve

(76) Inventor: William M. Appling, Granville, NY (US)

Correspondence Address: Harry K. Ahn, Esq., Reed Smith, LLP, 29th Floor, 599 Lexington Avenue, New York, NY, 10022, US

(21) Appl. No.: 10/465,501

(22) Filed: Jun. 19, 2003

Related U.S. Application Data
(60) Provisional application No. 60/390,166, filed on Jun. 19, 2002.

(51) Int. Cl. A61B 18/18

(52) U.S. Cl. 606/7

(57) ABSTRACT

An endovascular laser treatment device includes an optical fiber and a protective sleeve covering the optical fiber. The optical fiber and the protective sleeve are sized to be axially movable relative to one another between a protected state wherein the distal end of the optical fiber is protected within the sleeve and an operating state wherein the distal end of the optical fiber is outside of the sleeve. The optical fiber is in the protected state during insertion into the vessel or a sheath positioned within the vessel, and once it is inserted, the optical fiber is positioned in the operating state ready for application of laser energy to the target vessel.

### CROSS REFERENCE TO RELATED APPLICATIONS

This application claims priority under 35 U.S.C. §119 (e) to U.S. provisional application, Serial No. 60/390,166, filed Jun. 19, 2002, the disclosure of which is hereby incorporated by reference.

### FIELD OF THE INVENTION

The present invention relates to a medical device apparatus and method for treatment of blood vessels. More particularly, the present invention relates to a laser fiber device and method for endovenous thermal treatment of varicose veins.

### BACKGROUND OF THE INVENTION

Veins are thin-walled and contain one-way valves that control blood flow. Normally, the valves open to allow blood to flow into the deeper veins and close to prevent back-flow into the superficial veins. When the valves are malfunctioning or only partially functioning, however, they no longer prevent the back-flow of blood into the superficial veins. As a result, venous pressure builds at the site of the faulty valves. Because the veins are thin walled and not able to withstand the increased pressure, they become what are known as varicose veins which are veins that are dilated, tortuous or engorged.

In particular, varicose veins of the lower extremities is one of the most common medical conditions of the adult population. It is estimated that varicose veins affect approximately 25% of adult females and 10% of males. Symptoms include discomfort, aching of the legs, itching, cosmetic deformities, and swelling. If left untreated, varicose veins may cause medical complications such as bleeding, phlebitis, ulcerations, thrombi and lipodermatosclerosis.

Traditional treatments for varicosities include both temporary and permanent techniques. Temporary treatments involve use of compression stockings and elevation of the diseased extremities. While providing temporary relief of symptoms, these techniques do not correct the underlying cause, that is the faulty valves. Permanent treatments include surgical excision of the diseased segments, ambulatory phlebectomy, and occlusion of the vein through chemical or thermal means.

Surgical excision requires general anesthesia and a long recovery period. Even with its high clinical success rate, surgical excision is rapidly becoming an outmoded technique due to the high costs of treatment and complication risks from surgery. Ambulatory phlebectomy involves avulsion of the varicose vein segment using multiple stab incisions through the skin. The procedure is done on an outpatient basis, but is still relatively expensive due to the length of time required to perform the procedure.

Chemical occlusion, also known as sclerotherapy, is an in-office procedure involving the injection of an irritant chemical into the vein. The chemical acts upon the inner lining of the vein walls causing them to occlude and block blood flow. Although a popular treatment option, complications can be severe including skin ulceration, anaphylactic reactions and permanent skin staining. Treatment is limited to veins of a particular size range. In addition, there is a relatively high recurrence rate due to vessel recanalization.

Endovascular laser therapy is a relatively new treatment technique for venous reflux diseases. With this technique, the laser energy is delivered by a flexible optical fiber that is percutaneously inserted into the diseased vein prior to energy delivery. An introducer catheter or sheath is typically first inserted into the saphenous vein at a distal location and advanced to within a few centimeters of the saphenous-femoral junction of the greater saphenous vein. Once the sheath is properly positioned, a flexible optical fiber is inserted into the lumen of the sheath and advanced until the fiber tip is near the sheath tip but still protected within the sheath lumen.

Prior to laser activation, the sheath is withdrawn approximately 1-4 centimeters to expose the distal tip of the optical fiber. For proper positioning, a medical tape is conventionally used to pre-measure and mark the optical fiber before insertion into the sheath. The physician measures the sheath length and then marks the fiber with the tape at a point approximately 1-4 centimeters longer than the overall sheath length. This measurement is used to establish correct placement of the fiber tip relative to the sheath in an exposed position.

After the fiber tip has been exposed the correct distance beyond the sheath tip, the sheath and fiber are fixed together by tape or other means to hold the fiber in position relative to the sheath. The laser generator is then activated causing laser energy to be emitted from the bare flat tip of the fiber into the vessel. The energy contacts the blood causing hot bubbles of gas to be created. The gas bubbles transfer thermal energy to the vein wall, causing cell necrosis and eventual vein collapse. With the laser generator turned on, the optical fiber and sheath are slowly withdrawn as a single unit until the entire diseased segment of the vessel has been treated.

A typical laser system uses a 600-micron optical fiber covered with a thick polymer jacket. The fiber extends unprotected from the polymer jacket, approximately 4 mm in length at the tip of the optical fiber. The fiber's tip is ground and polished to form a flat face at its extreme distal end. The flat face is necessary to ensure energy is directed in a forward direction rather than radially, which would occur if the fiber tip configuration were radiused. The flat face of the optical fiber tip directs the laser energy from the fiber to the vein's lumen rather than directly to the vein walls.

The flat face of the fiber tip creates very sharp edges at the outer edge of the face. The optical fiber is bare at the tip and has no polymer jacket covering the distal most 4 mm section. There is no protection for the optical fiber's tip or for the internal wall of the sheath. When the sheath is advanced through the varicose vein, which is often tortuous, it assumes the curvature of the vein along its length. As the optical fiber is advanced through the sheath, the sharp edges inevitably contact the sheath's inside wall at curves in the sheath. As the optical fiber is advanced forward through the sheath lumen, the sharp edge of the optical fiber flat face contacts the sheath's inner wall at the outside of curves, causing shavings of the sheath material to be cut from the sheath wall. The shavings can be pushed ahead of the optical fiber as it is advanced through the sheath resulting in the shavings being left behind in the body. The shavings may be left to float freely in the venous system and will most likely become lodged in the pulmonary veins within the lung.

Another problem created by the current method is that optical fiber's tip may become damaged as it is being advanced through the curved, tortuous venous pathway of the sheath. Advancement may cause damage to the flat face ground at the optical fiber tip. Scratches or fractures in the optical fiber tip will cause energy to be refracted in variable directions resulting in possible perforation on the vein wall or incomplete closure of the diseased vein segment.

The prior art optical fiber and sheath designs also require the physician to pull back the optical fiber and sheath as a unit. The physician must be careful not to pull the optical fiber back inside the sheath during the laser procedure. If the laser were pulled inside the sheath while the laser energy was being delivered, the heat would damage the sheath. In the opposite scenario, if the sheath were pulled back without pulling the optical fiber, then too much energy would be delivered to a local area of the vein. The excessive energy would cause trauma possibly leading to perforations in the vein wall.

Therefore, it is desirable to provide an endovascular treatment device and method which protects the optical fiber tip during insertion into the sheath and which prevents the optical fiber from scraping the sheath's inner wall that may cause shavings of the sheath material to be introduced into the patient's venous system.

### SUMMARY OF THE DISCLOSURE

According to the principle of the present invention, an endovascular laser treatment device includes an optical fiber and a protective sleeve covering the optical fiber. The optical fiber and the protective sleeve are sized to be axially movable relative to one another between a protected state wherein the distal end of the optical fiber is protected within the sleeve and an operating state wherein the distal end of the optical fiber is outside of the sleeve. According to the invention, the optical fiber is in the protected state during insertion through a vessel or a sheath positioned within the vessel, and once it is inserted, the optical fiber is positioned in the operating state ready for application of laser energy to the target vessel.

According to the invention, the protective sleeve prevents the sharp edge of the optical fiber from contacting with and scraping against the inner wall of the vessel or the sheath. As a result, the present invention avoids any puncture of the vessel wall or sheath, and avoids creating any sheath shavings as the optical fiber advances through the sheath. Moreover, the protective sleeve advantageously protects the fiber tip from any damage as the device is being inserted through the vessel because the optical fiber is held stationary within the protective sleeve.

In another aspect of the invention, a switch is connected to the optical fiber and to the protective sleeve to provide positioning of the optical fiber tip. The switch has a protected position in which the optical fiber is in the protected state and an operating position in which the optical fiber is in the operating state. The movement of the switch from the protected position to the operating position causes longitudinal movement of the protective sleeve relative to the optical fiber so as to expose the optical fiber tip from the protective sleeve.

In another aspect of the invention, a method of using an endovascular laser treatment device is provided. A protective sleeve containing an optical fiber is inserted into a blood vessel. The optical fiber and the protective sleeve are axially movable relative to one another between a protected state wherein the distal end of the optical fiber is within the sleeve and an operating state wherein the distal end of the optical fiber is outside of the sleeve. Insertion of the protective sleeve is performed while the optical fiber is in the protected state. Once the protective sleeve is inserted into the blood vessel, the optical fiber in the protected state is positioned in the operating state to expose the distal tip of the optical fiber.

### BRIEF DESCRIPTION OF THE DRAWINGS

FIG. 1 is a plan view with a partial cross-section of the protective fiber assembly apparatus in the protected position.

FIG. 2 is a plan view with a partial cross-section of the protective fiber assembly apparatus in the operating position.

FIG. 3 is an enlarged view with a partial cross-section of the distal segment of the protective fiber assembly of FIG. 1.

FIG. 4 is an enlarged view with a partial cross-section of the distal segment of the protective fiber assembly of FIG. 2.

FIG. 5 is a plan view with a partial cross-section of the protective fiber assembly in the protected position coupled to an hemostasis introducer sheath.

FIG. 6 is a plan view with a partial cross-section of the protective fiber assembly in the operating position coupled to the hemostasis introducer sheath.

FIG. 7 is an enlarged view with a partial cross-section of the distal segment of the protective fiber assembly and hemostasis introducer sheath of FIG. 5.

FIG. 8 is an enlarged view with a partial cross-section of the distal segment of the protective fiber assembly and hemostasis introducer sheath of FIG. 6.

### DETAILED DESCRIPTION OF THE INVENTION

A preferred embodiment of the present invention is shown in FIGS. 1-8. The protective fiber assembly 1 shown in FIG. 1 includes a optical fiber 3, a protective sleeve 5, and a handle assembly 11 which also acts as a switch as will be explained in more detail below. As is well known in the art, the optical fiber 3 is typically comprised of a 600-micron laser fiber encased in a thick polymer jacket for the entire length of the fiber except for approximately 4mm at the distal end. The jacket prevents the fragile fiber from breaking during use. A thin intermediate cladding (not shown) creates a barrier through which the laser energy cannot penetrate, thus causing the energy to move longitudinally through the fiber 3 to the distal end where the laser energy is emitted. At the distal end, the optical fiber 3 extends unprotected from the polymer jacket.

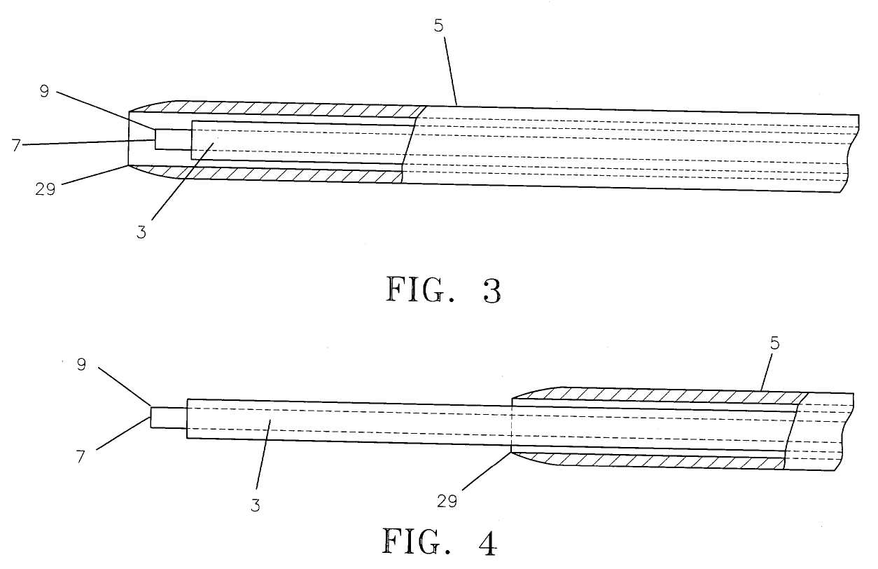

The proximal end of the optical fiber 3 is connected to a SMA 21 or similar-type connector, which can be attached to a laser generator (not shown). At the distal end, the optical fiber 3 tip is ground and polished to form a flat face 7 as shown in FIG. 3. The flat-faced surface 7 at the distal end of the optical fiber 3 ensures that laser energy is directed in a forward direction from the flat face 7 rather than radially, which would occur if the fiber tip configuration were radiused. Thus, the flat face 7 of the optical fiber 3 tip directs the laser energy from the fiber to the vein's lumen in a longitudinal direction rather than to the vein walls.

The retractable protective sleeve 5 provides protection to the unjacketed portion of optical fiber during insertion. The protective sleeve 5 is a tubular structure comprised of a flexible, low-friction material such as nylon. The sleeve 5 is arranged coaxially around the optical fiber 3. To accommodate the 600 micron optical fiber, the sleeve 5 inner diameter is preferably 0.045″, although other diameters can be used for different optical fiber sizes. The outer diameter of the protective sleeve 5 is sized to fit within a standard 5F sheath. Typically, a sleeve 5 dimensioned with a 0.066″ outer diameter should slidably fit within the lumen of a 5F sheath, which has an approximate inner diameter of 0.070″.

As shown in FIG. 3, the distal end of the protective sleeve 5 is radiused to facilitate insertion and advancement through the sheath. The proximal end of the protective sleeve 5 is securely attached to the distal handle component 13 of the handle assembly 11. Standard bonding methods are used to attach the sleeve 5 and distal handle component 13 together at point 25 as shown in FIG. 1.

The length of the sleeve 5 is dimensioned to ensure the sleeve tip 29 extends a few millimeters beyond the tip of the sheath when fully inserted, as shown in FIG. 4. Endovenous laser sheaths are typically 45 centimeters in length, although 60 and 65 centimeter sheaths are also well known in the art. The sleeve length is determined based on the length of the sheath being used for the procedure. According to the invention, the protective fiber assembly 1 can be sized to fit standard-length sheaths or custom-length sheaths. Further, the assembly 1 can be provided by itself or in a package that includes either the standard length sheath or custom-length sheath.

Turning now to the handle assembly 11 shown in FIG. 1, the assembly 11 is comprised of a distal handle component 13 and a proximal handle component 15. The two components are slidably connected with each other. Specifically, the distal handle component 13 is in coaxial arrangement with the proximal handle component 15, allowing for longitudinal movement between the two components relative to each other. Both handle components include through lumens, through which the optical fiber is positioned. The optical fiber 3 is securely attached to the proximal handle component 15 at a bond point 23. The sleeve 5, on the other hand, is attached to the distal handle component 13 at a fiber bond point 25 of the distal handle component 13.

Aside from being used as a handle, the handle assembly 11 is also a switch that controls longitudinal movement of the sleeve 5 relative to the optical fiber 3. The distal handle component 13 includes a longitudinally-positioned detent slot 17. A pin 19 attached to the proximal handle component 15 slides longitudinally within the detent slot 17 of the distal handle component 13.

The assembly 11 has two locking positions: protected position and operating position. When the pin is positioned in the proximal end detent position (protected position) 43 of the slot 17, the protective fiber assembly 1 is in a protected state. In that state, the distal end of the optical fiber 3 including the flat face 7 and sharp edges 9 are located within the lumen of the protective sleeve 5, as shown in FIGS. 1 and 3.

When locked into the proximal detent position (protected position) 43, the optical fiber 3 is held stationary in the protected state within the sleeve. When the protective fiber assembly 1 is advanced through a hemostasis introducer sheath 31 (see FIG. 5), the flat surface 7 and sharp edge 9 of the optical fiber 3 tip do not contact the sheath's valve gasket 41 and the sheath inner wall. Instead, the flexible sleeve 5 with its non-traumatic, tapered or radiused tip 29 comes in contact with the sheath's gasket 41 and inner wall.

According to the invention, the protective sleeve 5 serves three important advantages among others. First, the invention avoids any damage to the flat face 7 and sharp edge 9 of optical fiber 3 as the device is being inserted and advanced because the optical fiber 3 is held stationary within the protective tip 29 of the sleeve 5. Second, the protective sleeve 5 prevents the sharp edge 9 of the optical fiber 3 from contacting with and scraping against the inner wall of the sheath 33, which may create sheath shavings as the optical fiber 3 advances through the sheath. Third, because the sharp fiber tip does not come in contact with the sheath lumen during insertion, it allows the optical fiber to navigate through vein paths that are much more torturous than previously possible which permits the treating physician to treat the vessels that are located deeper in the body.

To prevent damage to the flat face 7 of the fiber during manufacture of the protective fiber device 1, the optical fiber 3 is preloaded into the sleeve 5/handle assembly 11. Damage to the optical fiber flat face 7 is prevented during assembly by inserting and advancing the optical fiber 3/proximal handle 15 assembly into the sleeve 5 while the sleeve is positioned in a straight, un-bent configuration. The straight, un-bent position ensures that there are no curves in the sleeve 5 during assembly. Inserting and advancing the optical fiber into the sleeve that is positioned straight with no curves prevents damage to both the sleeve 5 and fiber 3 during assembly.

In its final packaged state, the protective fiber assembly 1 is positioned in the retracted or protected position with the pin 19 in the proximal detent position 43 to ensure the integrity of the fiber tip during packaging and shipment. It also ensures that the device is in the correct, pre-treatment position when ready for use.

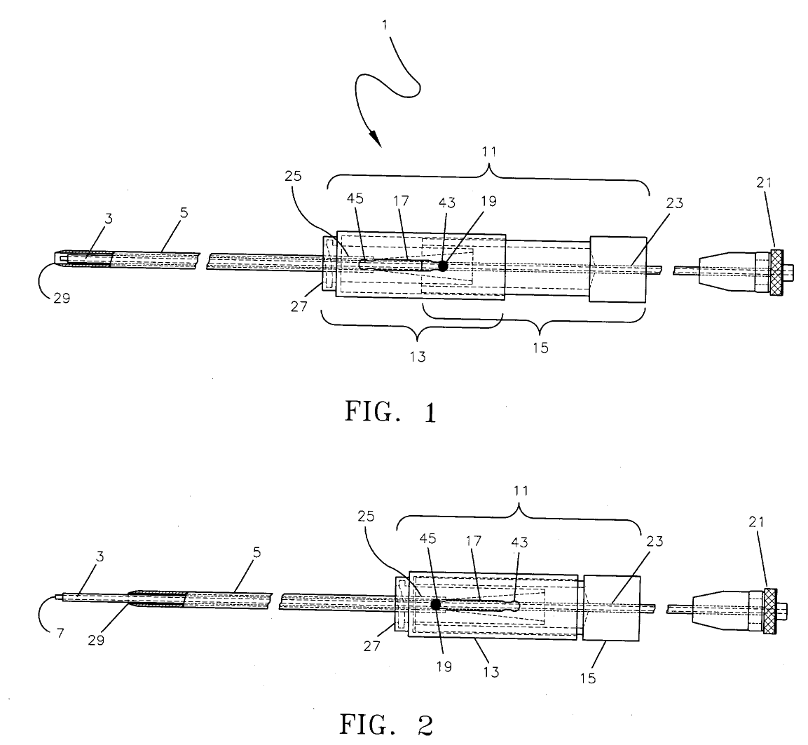

To expose the optical fiber tip 7, the distal handle component 13 is retracted relative to the proximal component 15. Preferably, the distal handle component 13 is retracted while the proximal component 15 is held stationary. This movement will cause the slot 17 to slide proximally until the pin 19 is positioned at the distal detent position 45, as shown in FIG. 2. Because the sleeve is securely attached to the distal handle component 13, retraction of component 13 results in a corresponding retraction of the sleeve 5. Since the optical fiber 3 is securely attached to the proximal handle component 15, which is held stationary during retraction, the optical fiber 3 remains stationary as the sleeve 5 is withdrawn, thus exposing the distal end of the optical fiber beyond the sleeve tip 29. FIG. 4 illustrates the position of the optical fiber 3 relative to the tip 29 of the sleeve 5 when the handle assembly 11 is in the retracted or operating position.

The handle mechanism 11 also controls the length of the exposed fiber 3. Specifically, the length of slot 17 is dimensioned to ensure that the optical fiber 3 tip extends beyond the sleeve tip 29 by the optimal length. Typically, the length of the slot is 2.5 centimeters, with a range of between 1 and 4 centimeters. The length of the slot 17 determines the length of the exposed fiber outside the sleeve 5 when the handle assembly 11 is moved to the retracted or operating detent position 45. The longitudinal dimension of the slot also controls the location of the optical fiber 3 distal end relative to the sleeve tip 29 when the handle assembly 11 is in the protected position with pin 19 located at proximal detent position 43.

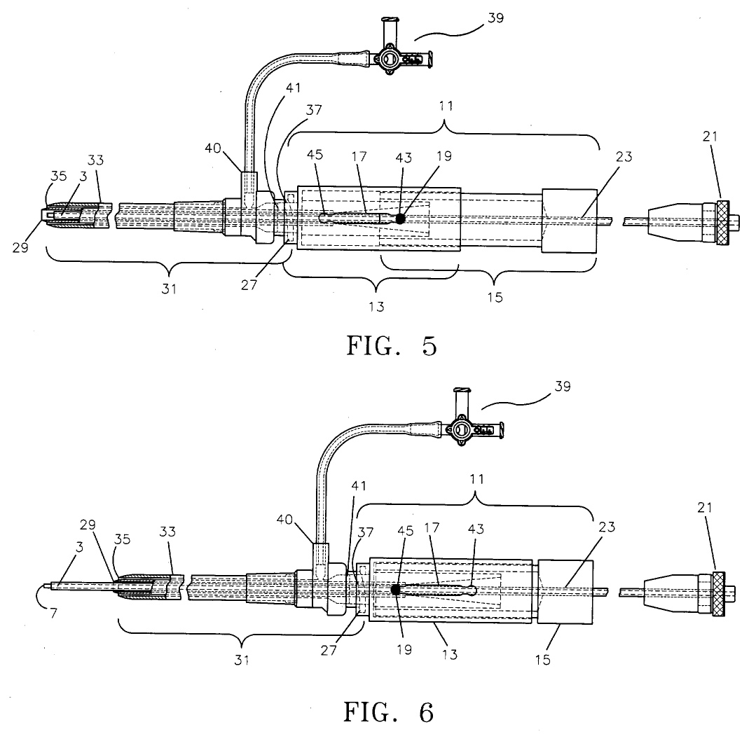

The handle mechanism 11 can be connected to a standard hemostasis introducer sheath as depicted in FIG. 5 and FIG. 6. The hemostasis introducer sheath assembly 31 is comprised of a sheath shaft 33, a sheath distal tip 35, side arm tubing and stopcock assembly 39, and a hemostasis valve gasket 41 housed within proximal opening of the sheath connection element (connector) 37. To connect the protective fiber assembly 1 to the hemostasis introducer sheath 31, the tip 29 of the protective sleeve 5 is inserted into and advanced through the sheath connection element 37 and sheath shaft 33 lumen until the handle connector 27 of the protective fiber assembly 1 comes into contact with the sheath connector 37. Threading the two connectors 27 and 37 together securely connects the protective fiber assembly 1 to the hemostasis introducer sheath assembly 31. A dual-thread arrangement, commonly used in medical devices, is shown in FIGS. 5 and 6, but other methods of connection are possible.

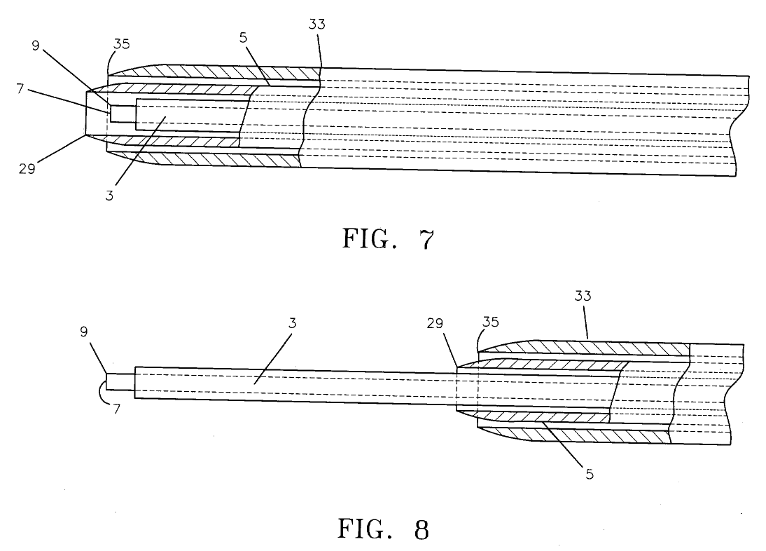

FIG. 5 shows the assembled protective fiber 1/hemostasis introducer sheath 31 with the handle assembly 11 in the protected detent position 43. FIG. 7 is an enlarged view of the distal end of the assembled protective fiber 1/hemostasis introducer sheath 31 showing the relative positions of the optical fiber 3, the protective sleeve 5 and the sheath shaft 33 when the device is in the protected position. In the embodiment shown, the flat face 7 of the optical fiber 3 is substantially aligned with the sheath 31 distal tip 35. The protective sleeve 5 distal tip 29 extends beyond the sheath tip 35 by a few millimeters. When assembled and in the protected position, the combined sheath tip 35/sleeve tip 29 configuration provides a radiused, non-traumatic profile for positioning within the vein.

FIG. 6 depicts the assembled protective fiber 1/hemostasis introducer sheath 31 with the handle assembly 11 locked into the operating position, as indicated by the position of pin 19 in the distal detent position 45. Because the hemostasis introducer sheath 31 is securely connected to the protective fiber assembly 1 by the connectors 27 and 37, retraction of the distal handle assembly 13 to the detent position 45 results in exposure of the optical fiber 3 as both the protective sleeve 5 and the hemostasis sheath 31 are retracted as a single unit.

A preferred method of using the protected fiber assembly 1 for treating varicose veins will now be described. The treatment procedure begins with the standard pre-operative preparation of the patient as is well known in the laser treatment art. Prior to the laser treatment, the patient's diseased venous segments are marked on the skin surface. Typically, ultrasound guidance is used to map the greater saphenous vein from the sapheno-femoral junction to the popliteal area.

The greater saphenous vein is accessed using a standard Seldinger technique. A small gauge needle is used to puncture the skin and access the vein. A guide wire is advanced into the vein through the lumen of the needle. The needle is then removed leaving the guidewire in place. A hemostasis introducer sheath as depicted in FIG. 5 is introduced into the vein over the guidewire and advanced to 1 to 2 centimeters below the sapheno-femoral junction.

The sheath includes a valve gasket 41 (FIG. 6) that provides a leak-proof seal to prevent the backflow of blood out the sheath proximal opening while simultaneously allowing the introduction of fibers, guidewires and other interventional devices into the sheath. The valve gasket 41 is made of elastomeric material such as a rubber or latex, as commonly found in the art. The gasket 41 opens to allow insertion of the optical fiber 3 and then seals around the protective sleeve 5 containing the optical fiber 3. However, the valve gasket 41 does not open in response to pressure from the distal side in order to prevent the back-flow of blood or other fluids. The gasket 41 also prevents air from entering the sheath through the proximal hub opening.

An inner dilator may be coupled with the hemostasis sheath to facilitate insertion and advancement of the sheath through the vein. Position of the sheath is then verified and adjusted if necessary using ultrasound. Once correct positioning is confirmed, the guide wire and dilator, if used, are removed leaving the sheath in place.

Procedural fluids may be flushed through the sheath lumen through the side arm stopcock/tubing assembly 39 coupled to the sheath through a side port 40. One commonly administered fluid during an endovascular laser treatment procedure is saline which is used to flush blood from the hemostasis sheath 31 prior to or after insertion of the protective sleeve 5 containing the optical fiber 3. Blood is often flushed from the sheath 31 to prevent the adherence of blood to the flat face 7 of the optical fiber 3, which can adversely affect the intensity and direction of the laser energy within the vessel. The sidearm tubing/stopcock 39 can also be used to administer emergency drugs directly into the vein.

The distal end of the protected fiber assembly 1 is then inserted into the hemostasis sheath 31 and advanced forward through the sheath 33 lumen. As the protected fiber assembly 1 is advanced through the curved pathway of the sheath shaft 33, the non-traumatic sleeve tip 29 rather than the sharp edge 9 of the optical fiber 3 comes in contact with the inner sheath wall. Advantageously, the sleeve tip 29 does not damage the inner wall of sheath shaft 33 as it is advanced because of the sleeve's flexible material characteristics as well as its tapered or radiused, non-traumatic distal profile. Moreover, the present invention eliminates the shavings of material that may be cut away from the inner wall of the sheath shaft 33 as a conventional unprotected fiber tip is advanced. Accordingly, there is no risk of shaft material being deposited within the venous system or becoming adhered to the flat face 7 of the optical fiber 3 when the protective fiber assembly 1 is used.

Because the optical fiber 3 is held in a stationary position within the sleeve, the fragile flat face 7 of the optical fiber 3 remains protected within the sleeve 5 and will not become marred or otherwise damaged during advancement through the sheath 33. This feature ensures that the laser energy is delivered to the vein in a forward rather than radial direction. Forward directed thermal energy is necessary to heat the blood sufficiently enough to create gas bubbles which in turn heat the vessel wall causing cell death and ultimately occlusion. Radially directed laser energy is emitted toward the vein wall instead of the blood, which may cause unintended perforation of the vessel wall and subsequently extensive bruising. Sleeve protection of the fiber flat face 7 also ensures that the integrity of the polished face surface is maintained so that a consistent level of thermal energy is delivered to the vein lumen.

The protected fiber assembly 1 is advanced through the sheath 31 until the sheath-connecting element 37 comes into contact with and can be threaded to the handle connector 27 of the fiber assembly 1. Once fully assembled, the combined protected fiber assembly 1/hemostasis sheath 31 appears as shown in FIG. 5 and FIG. 7. The handle assembly 11 is in the protected position as assembled during packaging, with the pin 19 in the proximal detent position 43. As shown in FIG. 7, the distal end of the fiber 3 is correctly aligned in the protected state within the sleeve 5 and sheath shaft 33 lumen when the protective fiber assembly 1 and sheath 31 are connected and the handle assembly 11 is in the protected position.

Once the treating physician has confirmed that the radiused sheath tip 35 is correctly positioned approximately 1-2 centimeters below the saphenous-femoral junction, the fiber tip 7 is automatically in the proper position as well, because the fiber tip is held in alignment with the sheath tip 35 axis by the proximal detent 43 locking feature of the handle assembly 11. Pre-measuring the sheath and taping or marking the fiber to identify the correct positioning is not required with the present invention. This handle locking feature also allows the physician to adjust the combined sheath 31/fiber assembly 1 position as a single unit without having to reposition the sheath 31 and fiber 3 separately. The protective fiber position is maintained during any required adjustments of the sheath.

Once the device is positioned within the vein, the tissue immediately surrounding the diseased vessel segment is subjected to numerous percutaneous injections of a tumescent anesthetic agent. The injections, typically lidocaine with or without epinephrine, are administered along the entire length of the greater saphenous vein using ultrasonic guidance and the markings previously mapped out on the skin surface. The tumescent injections perform several functions. The anesthesia inhibits pain caused from the application of laser energy to the vein. Secondly, the injection causes the vein to spasm, thereby reducing the diameter of the vein and bringing the vessel wall in close proximity to the optical fiber. The constricted vessel diameter facilitates efficient energy transmission to the vessel wall when the laser fiber is activated. The tumescent injection also provides a barrier between the vessel and the adjacent tissue and nerve structures, which restricts the heat damage to within the vessel and prevents non-target tissue damage.

Once tumescent injections have been administered, the device is placed in the operating position in preparation for the delivery of laser energy to the vein lumen. Specifically, the distal segment of the fiber 3 is exposed by retracting the connected distal handle component 13/sheath 31 hub while holding the proximal handle component 15 stationary. This movement causes the slot 17 of the distal handle component 13 to move proximally which causes the pin 19 to be repositioned from the protected detent position 43 (FIG. 5) to the operating detent position 45 (FIG. 6). As the distal handle component 13 is moved from the protected to the operating detent position, the sheath tip 35 and protective sleeve tip 29 are withdrawn as a single unit to expose the distal end of the optical fiber 3. Once fully retracted to the operating detent position 45, the fiber 3 extends beyond the sheath/sleeve tips 35 and 29 by approximately 2.5 centimeters.

The device 1 is now in the operating position, ready to delivery laser energy to the diseased vein. A laser generator (not shown) is connected to the SMA connector 21 of device 1 and is activated. The combined sheath 31/protective fiber assembly 1 is then slowly withdrawn together through the vein, preferably at a rate of 1-3 millimeters per second. The laser energy travels down the optical fiber 3, through the flat face 7 of the optical fiber and into the vein lumen, where it creates a hot bubble of gas in the bloodstream. The bubble of gas expands to contact the vein wall, along a 360-degree circumference, thus damaging vein wall tissue, and ultimately causing collapse of the vessel.

The laser energy should be directed forward in the bloodstream to create the bubble of gas. Having an undamaged, polished flat face 7 at the optic fiber distal tip is important to ensure that the laser energy is directed forward. Damage to the flat face 7 during introduction through the hemostasis sheath may result in laser energy being mis-directed radially against the vessel wall. Inconsistent delivery of laser energy may result in vessel wall perforations where heat is concentrated and incomplete tissue necrosis where insufficient thermal energy is delivered. The device of this invention avoids these problems by protecting the fiber flat face from damage prior to and during insertion into the sheath.

The threaded connection between the protected fiber assembly 1 and the sheath 31 hub ensures that the fiber tip 7 remains exposed beyond the sleeve tip 29 by the recommended length for the entire duration of the treatment procedure. Maintaining the optimal distance between the optical fiber tip and the sheath tip is necessary to avoid delivering energy to a non-targeted segment of the vessel. It is also necessary to ensure that the sheath tip is not in such close proximity to the fiber tip that thermal energy is inadvertently applied to the sheath causing damage. The device of the present invention prevents the user from inadvertently mis-positioning the fiber tip relative to the sheath tip by providing a simple, easy method of securely positioning and connecting the two components in optimal alignment without the use of ultrasound or other imaging techniques.

The procedure for treating the varicose vein is considered to be complete when the desired length of the greater saphenous vein has been exposed to laser energy. Normally, the laser generator is turned off when the fiber tip 7 is approximately 3 centimeters from the access site. The combined sheath 31/protective fiber assembly 1 is then removed from the body as a single unit.

The above description and the figures disclose particular embodiments of an endovascular laser treatment device with a protected sleeve. It should be noted that various modifications to the device might be made without departing from the scope of the invention. For example, the method of providing attachment of the connector and the hemostasis valve housing can be accomplished in many ways. The described embodiment depicts a dual thread arrangement, but methods such as snap fits or any other means for providing a secure but releasable connection could be used. Likewise, the described embodiment uses a pin within a slot to provide the control for the movement of the sheath and sleeve between the protected position and the operating position. The pin locks in a detent fashion at both ends of the slot. It should be noted that many other methods for providing such a controlled position adjustment could be used. For example, that same switch feature could be provided by a rotating sleeve (nut) and thread design where the sleeve could be rotated thereby retracting the sheath.

The diameter size of the optical fiber can also be modified. Although 600-micron diameter optical fibers are most commonly used in endovenous laser treatment of varicose veins, diameters as small as 200 microns, for example, can be used. With a smaller diameter optical fiber, the protective sleeve provides not only the functions previously identified above, but also increases the overall durability of the device. Specifically, the coaxially mounted sleeve provides added protection and strength to the fragile optical fiber.

What is claimed is:

1. An endovascular laser treatment device, comprising:
an optical fiber; and
a protective sleeve, the optical fiber positioned within the protective sleeve, the optical fiber and the protective sleeve being axially movable relative to one another between a protected state wherein the distal end of the optical fiber is within the sleeve and an operating state wherein the distal end of the optical fiber is outside of the sleeve;
the optical fiber being in the protected state during insertion into a vessel, the optical fiber being in the operating state once the optical fiber is positioned within the vessel.

2. The endovascular laser treatment device according to claim 1, further comprising a switch connected to the optical fiber and to the protective sleeve, the switch having a first position in which the optical fiber is in the protected state and a second position in which the optical fiber is in the operating state.

3. The endovascular laser treatment device according to claim 1, further comprising a sheath adapted to be inserted through the vessel, wherein the protective sleeve containing the optical fiber in the protected state is adapted to be inserted through the sheath positioned within the vessel.

4. An endovascular laser treatment device for use with a sheath, comprising:
an optical fiber; and
a protective sleeve, the optical fiber positioned within the protective sleeve, the optical fiber and the protective sleeve being axially movable relative to one another between a protected state wherein the distal end of the optical fiber is within the sleeve and an operating state wherein the distal end of the optical fiber is outside of the sleeve;
the optical fiber being in the protected state during insertion through the sheath, the optical fiber being in the operating state once the optical fiber is inserted through the sheath.

5. The endovascular laser treatment device according to claim 4, further comprising a switch connected to the optical fiber and to the protective sleeve, the switch having a first position in which the optical fiber is in the protected state and a second position in which the optical fiber is in the operating state, the movement of the switch between the first position and the second position causing longitudinal movement of the optical fiber relative to the protective sleeve.

6. An endovascular laser treatment device for use with a sheath inserted into a blood vessel, comprising:
an optical fiber;
a protective sleeve that receives the optical fiber and is sized to be inserted into a sheath; and
a switch attached to the optical fiber and the protective sleeve, the switch having a protected position where a distal end of the optical fiber is within the protective sleeve and an operating position where the distal end of the optical fiber is outside of the protective sleeve.

7. The endovascular laser treatment device according to claim 6, wherein the switch comprises:
a first component attached to the protective sleeve;
a second component attached to the optical fiber and coupled to the first component, the movement of the first component relative to the second component causing the switch to switch between the protected position and the operating position.

8. The endovascular laser treatment device according to claim 7, wherein the first component is slidably coupled to the second component and longitudinal movement of the first component relative to the second component causes the switch to switch between the protected position and the operating position.

9. The endovascular laser treatment device according to claim 8, wherein the switch is a detent switch having a longitudinal slot and a pin that slides within the longitudinal slot.

10. The endovascular laser treatment device according to claim 6, wherein the switch includes a distal connector adapted to securely connect to the sheath.

11. The endovascular laser treatment device according to claim 6, further comprising the sheath having a side port for connecting a side tube.

12. An endovascular laser treatment device for use with a sheath to treat varicose veins, comprising:
a sheath adapted to be inserted through a vessel;
an optical fiber;
a protective sleeve adapted to receive the optical fiber and sized to be inserted into the sheath; and
a switch attached to the optical fiber and the protective sleeve, the switch having a protected position where a distal end of the optical fiber is within the protective sleeve and an operating position where the distal end of the optical fiber is outside of the protective sleeve, the movement of the switch between the protected position and operating position causing longitudinal movement of the optical fiber relative to the protective sleeve.

13. The endovascular laser treatment device according to claim 12, wherein the switch includes a distal connector operable to securely connect to the sheath prior to switching of the switch to the operating position.

14. The endovascular laser treatment device according to claim 13, wherein when the switch is in the protected position and is securely connected to the sheath, the distal end of the optical fiber is substantially aligned with the distal tip of the sheath.

15. A method of using an endovascular laser treatment device, comprising:
inserting a protective sleeve containing an optical fiber through a blood vessel, the optical fiber and the protective sleeve being axially movable relative to one another between a protected state wherein the distal end of the optical fiber is within the sleeve and an operating state wherein the distal end of the optical fiber is outside of the sleeve, the inserting step being performed while the optical fiber is in the protected state; and
positioning the optical fiber in the operating state once the protective sleeve is positioned within the blood vessel.

16. The method according to claim 15, prior to the step of inserting a protective sleeve, further comprising inserting a sheath through the vessel wherein the step of inserting a protective sleeve includes inserting the protective sleeve and the optical fiber in the protected state through the sheath so as to prevent the distal end of the optical fiber from contacting the sheath wall.

17. The method according to claim 15, wherein:
the protective sleeve and the optical fiber are attached to a switch having a protected position associated with the protected state of the optical fiber and an operating position associated with the operating state of the optical fiber; and
the positioning step includes switching the switch from the protected position to the operating position.

18. The method according to claim 17, prior to the step of positioning, further comprising securely connecting the switch to the sheath.
